(19) United States
(12) Patent Application Publication
(10) Pub. No.: US 2025/0034530 A1
(43) Pub. Date: Jan. 30, 2025

(54) COMPOSITIONS AND METHODS FOR PRODUCTION AND USE OF A SCALABLE HUMAN CELL-DERIVED EXTRACELLULAR MATRIX

(71) Applicant: Ohio State Innovation Foundation, Columbus, OH (US)

(72) Inventor: Aleksander Skardal, Columbus, OH (US)

(21) Appl. No.: 18/716,634

(22) PCT Filed: Dec. 5, 2022
(86) PCT No.: PCT/US22/80875

§ 371 Date: Jun. 5, 2024

Related U.S. Application Data
(60) Provisional application No. 63/264,994, filed on Dec. 6, 2021.

(51) Int. Cl. C12N 5/09; C12N 5/077

(52) U.S. Cl. CPC C12N 5/0693; C12N 5/0656; C12N 2531/00; C12N 2533/54; C12N 2533/90; C12N 2537/10

(57) ABSTRACT

A method for preparing a human derived extracellular matrix (ECM) biomaterial are provided that include the steps of: preparing a hyaluronic acid and collagen microcarrier; adding to the hydrogel microcarrier human fibroblasts cells and a second cell line specific for driving fibroblast-based ECM secretion; culturing the microcarrier and cell mixture in a rotational wall vessel (RWV) reactor to form an organoid; decellularizing the resultant organoid to obtain an initial human derived ECM biomaterial. The human ECM-derived matrix prepared from this method is reliable, reproducible, and scalable and able support a variety of human cell lines.

### CROSS-REFERENCE

This application claims the benefit of U.S. Provisional Application No. 63/264,994, filed Dec. 6, 2021, which application is incorporated herein by reference in its entirety.

### FIELD

The human ECM-derived matrix prepared from this method is reliable, reproducible, and scalable and able support a variety of human cell lines.

### BACKGROUND

In vitro/ex vivo models of tissues and tumors, such as organoids and organ/tumor-on-a-chip platforms, have relied on animal tumor-derived extracellular matrix (ECM) biomaterials. While these materials are useful or research, they have drawbacks. First, the compositions are undefined consisting of a black box of cytokines, growth factors, mRNAs, and exosomes. Second, the conditions of use involve narrow temperature windows. Third, the materials show problematic lot-to-lot variability. Fourth, production is not scalable, requiring in vivo culture in tumor-bearing mice to obtain the material. Fifth, products such as Matrigel is derived from murine sarcomas, thus negating the advantage of a 3D ECM suitable for mimicking human disease. Moreover, as xenogeneic materials, they cannot be approved for scalable cGMP production of cells for human transplantation or tissue engineering.

It has also been shown that even after dissolving Matrigel to recover organoids, Matrigel based components contaminated recovered cellular material, confounding proteomic analysis. This renders Matrigel cultures cells inadequate for clinical applications.

A defined, bioengineered ECM hydrogel system to support a variety of human cell lines, and more importantly, patient-derived organoid (PDO) cultures. These PDOs can be formed from a wide variety of healthy tissues such as liver, heart, pancreas, adipose, and muscle, as well as malignant tumors, including colorectal, lung, appendiceal, melanoma, mesothelioma, sarcoma, glioma, and endocrine tumors. However, currently these platforms support establishment of these PDOs but long-term propagation and scalability of these platforms is very limited.

### SUMMARY

In this invention we provide a human ECM-derived matrix that is reliable, reproducible, and scalable and able support a variety of human cell lines, and more importantly, results in a wide array of patient-derived organoid (PDO) cultures. To this end, an approach to generating consistent batch-to-batch results in a new biomaterial for tissue engineering that is a consistent, human tumor ECM-derived matrix is provided. Applications of this matrix range from simple 2D cell cultures to 3D PDOs to organ- and tumor-on-a-chip systems, and even to patient-derived “avatar” xenograft.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE DRAWINGS

FIG. 1A-1E: Tumor cells induce fibroblasts to take on fibrotic phenotypes and secrete ECM. a) Fibroblasts cultured in conditioned media from the highly invasive CRC cell line HCT116 drastically take on an increased fibrotic morphology in 3D hydrogel cultures. Green—F-actin (phalloidin); Blue—DAPI. b) Likewise, fibroblasts under these conditions deposit significantly more collagen. Statistical significance: * p<0.05.

FIG. 2A-2E: RWV-based organoid culture and analyses. a-c) RWV culture. a-b) In an RWV bioreactor, cells and hydrogel microcarriers are combined and maintained in low fluid sheer stress rotational culture, resulting in c) organoids formed from cells adhering to microcarriers. Shown are organoid comprised of red fluorescence labeled HCT116 CRC cells and unlabeled mesenchymal stromal cells. Aliquots of organoids are removed and assessed by d) LIVE/DEAD staining to assess viability. Shown are organoids subjected to increasing concentrations of a toxic drug. Additionally, picrosirius red staining and polarized light microscopy will be used to query the relative amount of deposited collagen within organoids

FIG. 3A3-D: ECM fiber architecture. a) ECMs (PSR staining) represent dense, increased ECM deposition with b) increased alignment. c) Disorganized ECMs with less ECM deposition (small green fibers) with d) decreased alignment.

FIG. 4: Functionalization of adhesion proteins—laminin and fibronectin—and incorporation into 3D tissue constructs drives phenotype. Stellate cells are shown via macro-confocal microscopy following staining with phalloidin (green) and DAPI (blue). The addition of thiolated laminin conserves an epithelial phenotype. The addition of thiolated fibronectin drives many cells toward a mesenchymal phenotype. A 1:1 ratio of laminin and fibronectin results in a mixed phenotype. Scale bars—75 or 150 μm.

FIG. 5: Rheological assessment of existing hydrogels and the hydrogel of the instant invention. Shear elastic modulus (G′) measurements of Matrigel, HyStem, HA-collagen, HA-Collagen mixed with Humagel™ and Humagel™. Statistical significance: *p<0.01.

FIG. 6: Biofabrication of tumor organoids. CRC cell lines or patient-derived tumor biospecimens are employed using human derived ECM biomaterial (or control hydrogel biomaterials) to bioprint tumor organoids for downstream analyses.

FIG. 7A-7C: Proliferation of cell lines in Humagel™ and other ECM-derived hydrogels. Quantification of ATP activity (Celltiter Glo 3D assay) over time in HyStem, HA (hyaluronic acid)-collagen, HA-collagen mixed with Humagel™, and Humagel™ in 3D cultures of HCT-116 CRC cells (7A), HT-29 CRC cells (7B), and A-172 glioblastoma cells (7C). Statistical significance: *p<0.05 between Humagel™ and other groups.

FIG. 8: A table outlining the protein composition of a Humagel™.

### DETAILED DESCRIPTION

As we have recognized that the extracellular matrix (ECM) within tissues is not only a physical scaffolding, but also a dynamic, evolving entity complete with cellular signaling cascade-inducing capabilities, minimalist biomaterials are inadequate for most tissue engineering applications. The ECM is crucial in directing cell differentiation, organization, and motility. Interestingly, most biomaterials employed in tissue engineering applications fall on one of two sides of a spectrum spanning from minimalist one- or two-component hydrogels to complex, “black box” ECMs derived from animal tissues, with relatively few in between. For example, on the minimalist side have been many efforts in 3D bioprinting where gelatin or gelatin-alginate hydrogel biolinks have been favored for a number of years due to handling efficiency and printing compatibility. Alginate is not present in human or animal ECM, and gelatin—as degraded collagen—represents but one component of the ECM. On the other side of the spectrum are animal-derived ECMs, such as Matrigel—the industry standard—which while incredibly potent with undefined growth factors, cytokines, and likely undefined miRNAs and exosomes, are non-starters for translational efforts that require eventual FDA approval to move towards clinical use, either for direct use in patients or as patient-specific diagnostic tools. As such, while products such as Matrigel are quite ubiquitous today, such products inherently lack the ability to capture significant market share as translational and clinical products.

To overcome these limitations, this invention describes a human derived ECM biomaterial that is reliable, reproducible, and scalable. We have developed an approach to generating batch-to-batch consistent, human tumor ECM, as a new biomaterial for tissue engineering. Applications of the human derived ECM biomaterial range from simple 2D cell cultures to 3D PDOs to organ- and tumor-on-a-chip systems, and to patient-derived “avatar” xenograft models. In some aspects, a tumor cell line support consistent batch to batch in terms of ECM composition. Similarly, tissue, but not decellularized tissue may be use of ECM formation, followed by decellularizing methods at the harvest stage.

Aspects of the invention are based on rotating wall vessel (RWV) bioreactors in which large numbers of cells can be cultured on biomaterial microcarrier beads; this allows production of thousands of spherical organoids in a limited culture volume. Importantly, the RWV is scalable from a simple 50-mL laboratory size to 1-L, 5-L, or about 10 L, or about 50 L, or about 100 L, or larger size for production at scale. With this method, we employ high density human immortalized cell line and fibroblast cocultures, in which secreted cytokines induce activation of fibroblasts and subsequent ECM deposition. This ECM will be processed to obtain a human-derived ECM biomaterial which provides a defined, human-specific tumor ECM biomaterial product compared to current tissue-derived ECM products. An exemplary ECM biomaterial derived from colorectal cancer (CRC) cells to from an ECM-biomaterial-CRC is described in the examples.

In some aspects, the invention provides a microcarrier. The microcarrier may comprise a collagen, collagen analog, collagen mimetic, a partial collagen fragment, a modified collagen, a gelatin, a methacrylated gelatin, collagen or hyaluronic acid, a thiolated collagen, gelatin, or hyaluronic acid, a maleimide modified gelatin, collagen or hyaluronic acid or any combination of these molecules.

In some aspects, the method for preparing a human derived extracellular matrix biomaterial may include culturing a microcarrier with a stromal cell. In some aspects this stromal cell is a fibroblast. It is appreciated however that cell such as stellate cells, smooth muscle cells, astrocytes or any other tissue specific stromal cell that is efficient for secreting an extracellular matrix are usable in the methods for preparing a human derived extracellular matrix biomaterial described herein.

In some aspects, the adhesion protein may include any modified fibronectin, laminin, collagen III/IV or proteoglycan. In some aspects the modification may include thiolation, methacrylation, or maleimide modified adhesion proteins.

In some aspects, this method provides a human tumor specific ECM employing high density human tumor cell line and fibroblast cocultures, in which tumor-secreted cytokines induce activation of fibroblasts and subsequent ECM deposition. This human derived ECM biomaterial is a defined, human-specific tumor ECM biomaterial product in comparison to current tissue-derived ECM products. Importantly, by varying the human tumor cell line employed, we can establish a portfolio of products from different tumor types and grades. The products provide human tumor ECMs that would be suitable for laboratory, preclinical, and clinical research, such as PDOs or tissue constructs for transplantation.

In some aspects, methods for preparing a human derived extracellular matrix (ECM) biomaterial are provide that include the steps of: preparing a hyaluronic acid and collagen microcarrier; adding to the hydrogel microcarrier human fibroblasts cells and a second cell line specific for driving fibroblast-based ECM secretion; culturing the microcarrier and cell mixture in a rotational wall vessel (RWV) reactor to form an organoid; decellularizing the resultant organoid to obtain an initial human derived ECM biomaterial; and processing and purifying the initial human derived ECM biomaterial to obtain a human derived ECM biomaterial.

In some aspects, the hyaluronic acid and collagen microcarrier to be used in the method is prepared by a process including the steps of: combining thiolated hyaluronic acid and methacrylated collagen with a dextran bead; cross-linking the mixture, and lyophilizing followed by sterilizing the mixture. In some aspects, the method may additionally include the addition of a thiolated fibronectin, a thiolated laminin, or a combination thereof to the cell and microcarrier mixture. In some aspects, the RWV reactor used in the methods have a reactant volume that is between 50 mL, or 1 L, or 2 L or about 5 L, or about 10 L, or about 50 L, or about 100 L or greater.

In some aspects, the method of the invention is carried out with a second cell line is an established tumor cell line. In some aspects, the invention is carried out with a second cell that is an established organoid, or a patient derived cell line or organoid. A patient derived cell may be from healthy, disease-free tissues or may be a tumor or malignant cell.

In some aspects, the human derived ECM biomaterial produced from the methods of the invention may comprise characteristics specific for the second cell line.

In some aspects, the processing and purifying step of the method includes at least one process that is a lyophilizing, sterilizing, filtering or centrifuging step.

In some aspects, the invention comprises a panel of multiple unique human derived ECM biomaterials prepared according to the method of claim 1, each defined by a unique characteristic of the second cell line.

In some aspects, an improved scalable rotating wall vessel bioreactor is provided. In some aspects, the RWV organoid cocultures of a high density human immortalized cell line and fibroblast cocultures is provided. From the cocultures, secreted cytokines induce activation of fibroblasts and ECM depositions. A cell-free ECM is harvested and processed from the RWV to obtain a defined human-specific tumor ECM biomaterial.

In some aspects, the invention may also encompass a human derived ECM biomaterial having a hyaluronic acid and collagen microcarrier, and at least one fibroblast or second cell secreted molecule including one of: collagen, glycosaminoglycans, elastin, fibronectins, laminins, growth factors, or cytokines.

In yet another aspect, a method of preparing an organoid of a patient derived tissue or tumor is provided. The method would include steps of: providing a defined human derived ECM biomaterial, adding to the biomaterial cells of the tissue or tumor to be cultured, culturing the biomaterial and cells in a rotational wall vessel (RWV) reactor for a specified period of time to form an organoid specific for the patient derived tissue or tumor.

### Definitions

### Abbreviations

As used herein and in the appended claims, the singular forms “a”, “and”, and “the” include plural referents unless the context clearly dictates otherwise. Thus, for example, reference to “a sample” also includes a plurality of such samples and reference to “the splicing regulator protein” includes reference to one or more protein molecules, and so forth.

As used herein, the term “about” refers to +/−1% deviation from the basic value.

As used herein, the term “tumor” refers to any neoplastic growth, proliferation, or cell mass whether benign or malignant (cancerous), whether a primary site lesion or metastases.

Unless defined otherwise, all technical and scientific terms used herein have the same meaning as commonly understood by one of ordinary skill in the art to which this invention belongs.

### EXAMPLES

### Example 1-Tumor Driven Fibroblast-Deposition of ECM In Vitro

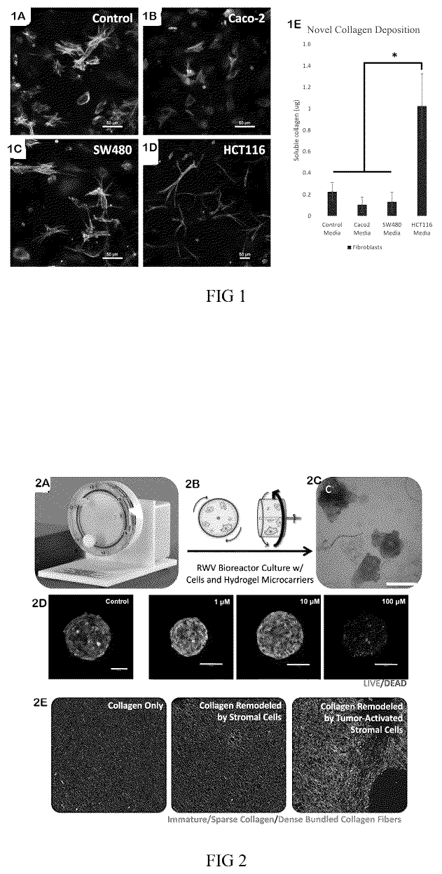

Rather than utilizing animal models and animal tumors, to generate potent ECMs, we will employ human-based tumor organoids. Organoids comprised of a hyaluronic acid (HA) and collagen hydrogel, a HCT116 CRC tumor cells line, and fibroblasts are provided. Referring now to FIG. 1A, these organoids generate increased mesenchymal phenotype in the embedded fibroblasts (FIG. 1A) and a significantly enhanced level of ECM deposition (FIG. 1B). In FIG. 1A, Fibroblasts cultured in conditioned media from the highly invasive CRC cell line HCT116 drastically take on an increased fibrotic morphology in 3D hydrogel cultures. Green represents F-action (phallodin) and Blue-DAPI. Likewise, in FIG. 1B, fibroblasts under these conditions deposit significantly more collagen (Statistical significance: *p<0.05).

Static culture of tumor organoids in well plates is difficult to scale for production of ECM materials for commercial use. Therefore, in some aspects, the methods may be used with RWV bioreactor cultures of organoids to generate and maintain a significantly increased organoid volume to media volume in culture. These cultures include a hydrogel microcarrier technology and specifically support tumor organoids in RWV culture. Now, this will be the first use of this microcarrier technology to act as receptacles for tumor-induced ECM product.

Use of tumor cell lines may support consistency batch-to-batch in terms of ECM composition.

### Example 2: Synthesis of ECM Hydrogel Microcarriers for RWV Culture

Microcarriers are synthesized with hyaluronic acid (HA) and collagen hydrogel. Inclusion of collagen has been beneficial in supporting matrix remodeling in tissue constructs and organoids. Thiolated HA and methacrylated collagen are dissolved at 2 mg/mL and 6 mg/mL, respectively, using water and a neutralization solution, both containing 0.1% w/v photoinitiator. The HA and collagen solutions are then mixed in a 3:1 ratio prior to immediate use. To aid in driving mesenchymal phenotypes of cells, 0.05 mg/mL of thiolated fibronectin (synthesized in house) is incorporated. The hydrogel precursor solution (10 mL) is then added to 0.5 g dextran Sephadex G-50 beads. As the beads swell with the introduction of the hydrogel solution, the hydrogel solution is pulled into the pores of the beads. The resulting “paste-like” material is spread out on foil or plastic and crosslinked either by pH driven thiol-methacrylate crosslinking (˜20 minutes) or UV irradiation-driven thiol-ene crosslinking (nearly instantaneous). The resulting materials is then crumbled into conical tubes and lyophilized.

Prior to cell culture, microcarriers are sterilized in PBS by autoclaving at 115° C. for 15 min. The sterilized beads can then be stored at 4° C. Cells and microcarriers in medium (DMEM) will be added to a 50 mL RWV bioreactor (Synthecon, Houston, TX) to reach a final density of 80,000 cells/5 mg beads/mL medium with a ratio of 1:1 HCT116: fibroblasts (human fibroblasts cell line BJ, ATCC). Rotation of the RWV bioreactors will be started immediately and continued for 14 to 21 days. Medium will be first changed on day 5 of culture in order to allow the cells to bind to the beads, after which medium will be changed every 1 or 2 days. The RWV bioreactors are generally set to initially rotate at 18 rpm; the rate of rotation will be manually increased throughout culture to keep the clusters in “freefall” as they increase in size.

### Example 3: Organoid and ECM Analysis During Culture

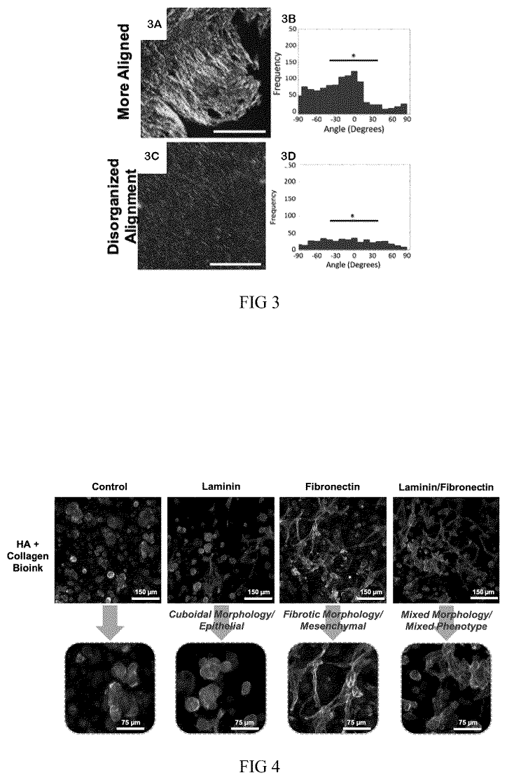

Aliquots containing organoids will be removed twice per week and used to assess growth through size quantification of light microscopy images (n of 10 or higher) and viability through LIVE/DEAD staining and fluorescent microscopy. We will then quantify collagen fiber alignment, width, and length to measure changes in architecture rather than composition. Tissue sections of organoids will be prepared, stained with picrosirius red (PSR), and imaged under polarized light to visualize collagen fibers (FIG. 2e). To quantify the PSR staining data, we use the CT-FIRE (Curvelet Transform plus FIRE algorithm) software platform. CT-FIRE allows automatically isolates and analyzes collagen fibers in an image and quantifies them with descriptive statistics, such as fiber angle, fiber length, fiber straightness, and fiber width (FIG. 3). RNA sequencing to evaluate cell population stability batch to batch. One important factor in ensuring product consistency and reproducibility will be verifying stability of the cells involved; in particular the tumor cells. Towards this end, we will generate at least 4 batches of organoids and subsequent ECM materials (i.e. multiple product lots). While the HCT116 cells line is a long-established cell line that researchers consider relatively stable, it is possible that these cells can experience genetic drift over time, which we wish to minimize. We will employ HCT116 cells from 4 different passage numbers, and perform RNAseq, analyzing the top 1000 most variably expressed genes, thereby evaluating the extent of genetic changes in the cell line. We will then isolate RNA (Qiagen Rneasy) and RNAseq will be performed (OSUCCC Genomic Shared Resource). These data will be trimmed, filtered and aligned using an R Bioconductor package. Cell clustering will be determined by principal component analysis and analyzed.

Modulation of the efficiency of deposition could be performed. In recent studies, we showed that modulation of hydrogel constructs containing stellate cells (liver stromal cells) could be driven to more mesenchymal or epithelial phenotypes by incorporation of thiolated fibronectin or laminin, respectively (FIG. 4).

### Example 4: Decellularize Organoids and Isolate and Characterize Organoid-Derived ECM Composition and Gelation Properties

Once a human derived ECM biomaterial product is formed from the batches of organoids cultured in the RWV bioreactors processing steps to isolate the ECM components from the organoids are performed after which we will perform the necessary characterization studies and hydrogel gelation and mechanical testing to verify the usability of the human derived ECM biomaterial product.

Organoid decellularization, or cell removal. Organoids will first be rinsed with chilled Dulbecco's phosphate buffered saline (DPBS), after which they will be transferred to distilled water and shook on a rotary shaker at 200 rpm for 24 hours at 4° C., during which water will be changed three times. The organoids will then be treated with 2% Triton X (TX)-100 for 24 hours followed by 2% TX-100+0.1% NH4OH for 24 h. During the TX-100 rinses, solutions will be changed twice daily. The cell free organoid tissues will be washed for 24 additional hours in distilled water to remove any traces of TX-100, after which can be stored at 4° C. until further use.

ECM dissolution. Acellular organoid ECMs will be lyophilized for 48 h. Following lyophilization, samples will be ground into a powder with a freezer mill (or mortar and pestle). One gram of ECM powder will be mixed with 100 mg Pepsin (Porcine gastric mucosa, 3400 units of protein) and sterilized by gamma irradiation (1 Mrad). All subsequent procedures following sterilization are then carried out under sterile conditions. Hydrochloric acid (0.1 N, 10 mL) will be added to the sterilized materials and incubated for 48 h at room temperature. The resulting mixture will be transferred to a conical tube and centrifuged at 3000 rpm for 15 min. The supernatant will be reserved and the pellet was discarded. This is repeated generally 3 times until the supernatant is clear. The suspension is filtered through a 0.2 μm syringe filter (Fisher Scientific). The decellularized ECM extract can be stored at −80° C.

ECM product composition analysis. Tocharacterize ECM component levelsbatch-to-batch ECM components will be measured by BioColor assay kits (Collagen, Glycosaminoglycans, and Elastin) and ELISA (Fibronectin, Laminin).34,36,38 In addition, ECM solutions will be evaluated for a panel of 440 cytokines, including many key growth factors, using cytokine arrays (RayBiotech).

Gelation and rheological testing with and without de-lipidation. Aliquots of ECM solutions will be used as is, or de-lipidated. Removal of remaining lipids will be performed using established protocols utilizing ethanol. Following de-lipidation steps, cold ECM solutions in glass vials will be brought to pH 7.0-7.4 using weak NaOH and allowed incubate at 37° C. for 20, 40, and 60 min to induce hydrophobic physical crosslinking. At these timepoints, bulk gelation will be assessed by inverting each vial and observing whether the ECM material remains in the inverted bottom of the vials or if it flows. In addition, gelation will be evaluated quantitatively through rheological testing. Aliquots of the ECMs will be prepared as described and pipetted onto the plate of our TA Instruments Discovery HR-2 rheometer using a plate and plate test bed with an 8 mm diameter geometry in order to utilize smaller aliquot volumes. At 0, 20, 40, and 60 min, ECMs will undergo mechanical testing by rheology (stress sweeps and micro-compression testing, TA Instruments DHR-2) using methods we have extensively published. Using these established testing protocols, the rheometer allows for evaluation of stiffness, viscosity, and mechanical properties of fluids and viscoelastic materials such as tissue and biomaterials.

Referring to FIG. 5, we have performed rheology studies on the ECM hydrogel materials of the instant invention (Humagel™) without de-lipidation, and compared shear elastic modulus values (G′) to those of Matrigel, Hystem, and HA-collagen.

### Example 5: Evaluate Cell Viability and Proliferation in Humagel™-CRC Versus Murine Sarcoma-Derived Hydrogels

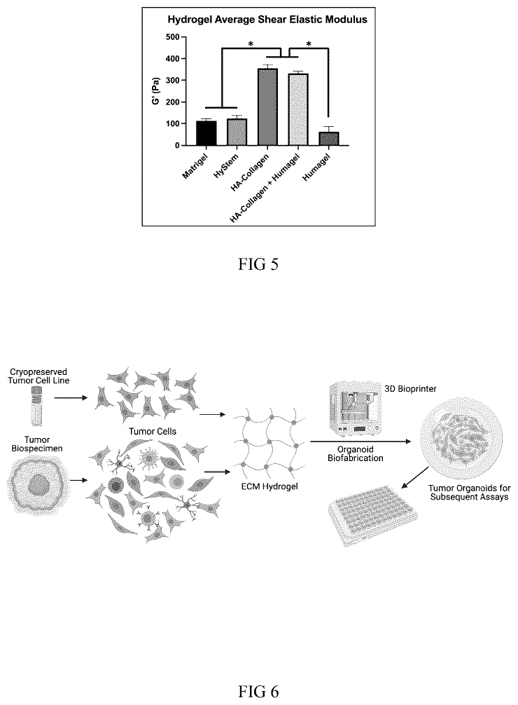

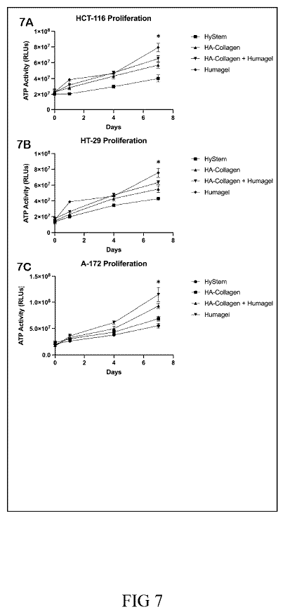

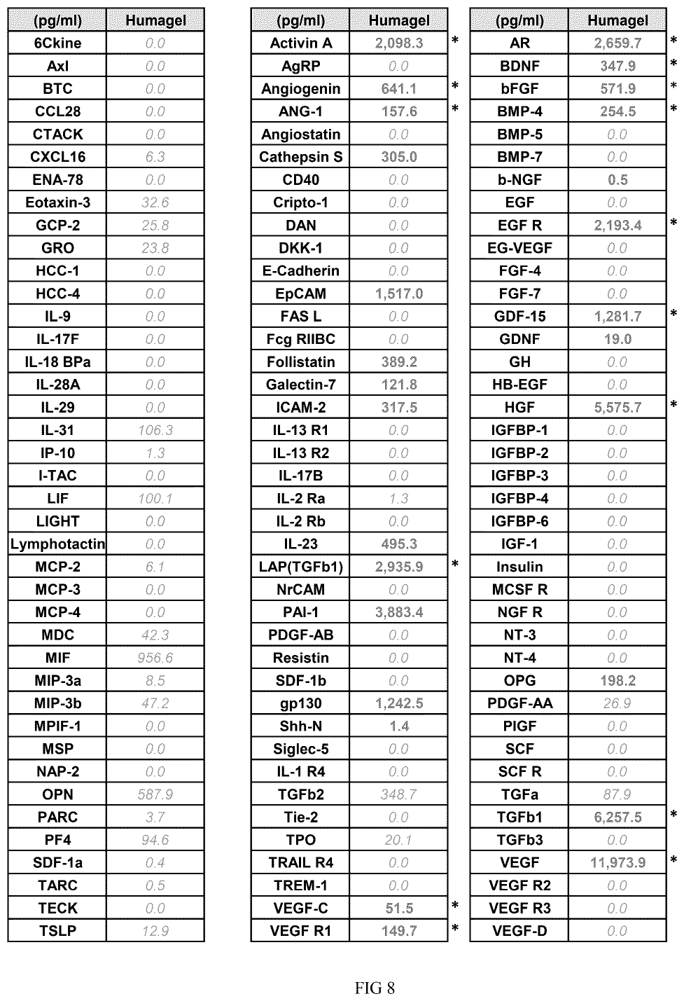

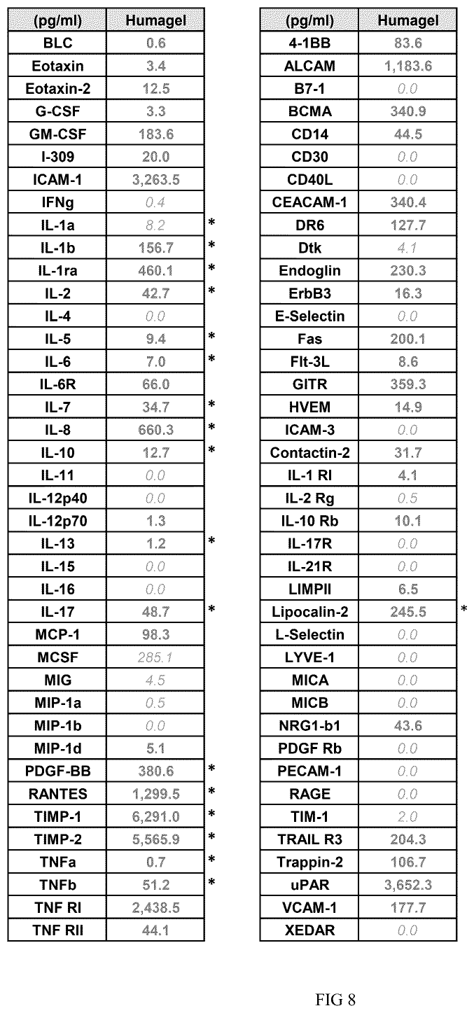

CRC organoid bioprinting. HCT116, SW480, and Caco2 CRC cells will be suspended in the Humagel™ solution at 20 million cells/mL and bioprinted (Cellink Bio X) in 10 μL volumes into 96-well plates (FIG. 6). In parallel, our HA-collagen hydrogel (same formulation as that used to create microcarriers) and Matrigel will also be used as control groups to bioprint cell line tumor organoids as described previously. Viability and proliferation of cells within organoids will be determined on days 1, 4, and 7 following organoid biofabrication. Viability will be determined by LIVE/DEAD staining and fluorescent imaging. Proliferation will be assessed using ATP activity quantification (CellTiter Glo 3D, Promega). Specifically, we will focus on the magnitude of increases in ATP activity timepoint to timepoint. Positive increases indicate proliferation of the cells within the organoids. Deidentified CRC biospecimens have already been procured in cooperation with the Ohio State University Comprehensive Cancer Center Tissue Procurement Core, and following mincing, were banked for use in a variety of organoid studies. Biospecimens will be thawed, washed, and incubated with collagenase/hyaluronidase to digest the ECM. Portions will be preserved for histology and RNA extraction. Subsequent cell suspensions undergo dead cell removal and filtration prior to use in PTOs. PTOs will be biofabricated as previously described in a similar manner as the cell line organoids described above. Viability and proliferation will be assessed as described above. In addition to human derived ECM biomaterial and HA-collagen hydrogels, Matrigel will be used as a third ECM for comparison. In PTO cultures using our HA-collagen hydrogels, we observe great viability, but often proliferation is limited. Proliferation assays will specifically focus on improvements over the ECM/growth factor free HA-collagen hydrogel. As expected, 1) Cell line tumor organoids with viability >95% and increased ATP activity levels from timepoint to timepoint. 2) Establishment of viable CRC PTOs from at least 3 separate biospecimens, with a viability of at least 80% after 7 days in culture, and statistically significant increases in proliferation compared to culture in HA-collagen hydrogels (without ECM supplementation). 3) Comparable or better viability and proliferation metrics versus Matrigel PTOs occur. Referring to FIG. 7, Humagel™ outperforms other of the hydrogels for proliferation of two CRC tumor cell lines and a glioblastoma cell line. Matrigel was omitted solely due to supply-chain issues resulting in the product remaining unavailable for several months.

### Example 6: Protein Composition of Humagel™

Referring now to FIG. 8, a table demonstrating the detectable protein profile of the Humagel™ product described in the invention. In FIG. 8, asterisks denote proteins known to be potent in a number of biological functions such as migration, chemotaxis, cancer progression, angiogenesis, inflammation, etc.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A method for preparing a human derived extracellular matrix (ECM) biomaterial comprising:
preparing a microcarrier;
adding to the microcarrier human stromal cell characterized by secreting an ECM and a second cell line specific for driving the stromal cell-based ECM secretion;
culturing the microcarrier and cell mixture in a rotational wall vessel (RWV) reactor to form organoids;
decellularizing the resultant organoid to obtain an initial human derived ECM biomaterial;
processing and purifying the initial human derived ECM biomaterial to obtain a human derived ECM biomaterial.

2. The method of claim 1, wherein the microcarrier is prepared by a process comprising:
combining thiolated hyaluronic acid and methacrylated collagen with a dextran bead;
cross-linking the mixture, and
lyophilizing followed by sterilizing the mixture.

3. The method of claim 1, wherein the human stromal cell is a fibroblast cell, a stellate cell, a smooth muscle cell, an astrocyte, or any stromal cell efficient at secreting extracellular matrix.

4. The method of claim 1, wherein the microcarrier comprises any combination of a collagen, collagen analog, collagen mimetic, a partial collagen fragment, a modified collagen, a gelatin, a methacrylated gelatin, collagen or hyaluronic acid, a thiolated collagen, gelatin or hyaluronic acid, a maleimide modified gelatin, collagen or hyaluronic acid.

5. The method of claim 1, further comprising adding during the adding step a modified adhesion protein.

6. The method of claim 5, wherein the modified adhesion protein comprises a thiolated, methacrylated or maleimide modified fibronectin, laminin, collagen III/IV, proteoglycan, or any combination thereof.

7. The method of claim 1, wherein the RWV reactor comprises a reactant volume that is between about 50 mL and about 5 L, or about 10 L, or about 50 L, or about 100 L.

8. The method of claim 1, wherein the second cell line is an established tumor cell line.

9. The method of claim 1, wherein the second cell line is an established organoid.

10. The method of claim 1, wherein the second cell line is a patient derived cell or organoid.

11. The method of claim 1, wherein the human derived ECM biomaterial comprises characteristics specific for the second cell line.

12. The method of claim 1, wherein processing and purifying the initial human derived ECM biomaterial comprises at least one process of lyophilizing, sterilizing, filtering or centrifuging.

13. A panel of multiple unique human derived ECM biomaterials prepared according to the method of claim 1, each defined by a unique characteristic of a unique second cell line.

14. A human derived ECM biomaterial comprising:
a hyaluronic acid and collagen microcarrier,
at least one fibroblast or second cell excreted molecule including one of: collagen, glycosaminoglycan, elastin, fibronectins, laminins, growth factors, or cytokines.

15. The human derived ECM biomaterial of claim 14 further comprising a protein that is activin A, Angiogenin, ANG-1, Cathepsin S, EpCAM, Follistatin, Galectin-7, ICAM-2, IL-23, LAP, PAI-1, gp130, Shh-N, VEGF-C, VEGF R1, AR, BDNF, bFGF, BMP-4, b-NGF, EGF R, GDF-15, GDNF, HGF, OPG, TGFb1, VEGF, BLC, Eotaxin, Eotasxin-2, G-CSF, GM-CSF, I-309, ICAM-1, IL-1b, IL-1ra, IL-2, IL-5, IL-6, IL-6R, IL-7, IL-8, IL-10, IL-12p70, IL-13, IL-17, MCP-1, MIP-1d, PDGF-BB, RANTES, TIMP-1, TIMP-2, TNF-A, TNGB, TNF RI, TNG RII, 4-1BB, ALCAM, BCMA, CD14, CEACAM-1, DR6, Endoglin, ErbB3, Fas, Flt-3L, GITR, HVEM, Contactin-2, IL-1 RI, IL-10 Rb, LIMPII, Lipocalin-2, NRG-b1, TRAIL R3, Trappin-2, uPAR, or VCAM-1.

16. A method of preparing an organoid of a patient derived tissue or tumor, the method comprising:
providing a defined human derived ECM biomaterial prepared according to claim 1,
adding to the biomaterial cells of the tissue or tumor to be cultured,
culturing the biomaterial and cells in a rotational wall vessel (RWV) reactor for a specified time to form an organoid specific for the patient derived tissue or tumor.
